(19) United States
(12) Patent Application Publication
(10) Pub. No.: US 2023/0165838 A1
(43) Pub. Date: Jun. 1, 2023

(54) TREATMENT FOR ACUTE RESPIRATORY DISTRESS SYNDROME

(71) Applicant: DIMERIX BIOSCIENCE LTD., Nedlands (AU)

(72) Inventors: Nina Webster, Strathmore (AU); Robert Pearson Shepherd, Upwey (AU)

(21) Appl. No.: 17/921,897

(22) PCT Filed: May 3, 2021
(86) PCT No.: PCT/AU2021/050404

§ 371 Date: Oct. 27, 2022

Related U.S. Application Data
(60) Provisional application No. 63/024,348, filed on May 13, 2020.
(60) Provisional application No. 63/021,084, filed on May 6, 2020.

(51) Int. Cl. A61K 31/4184; A61K 31/28; A61P 11/00

(52) U.S. Cl. CPC A61K 31/4184; A61K 31/28; A61P 11/00

(57) ABSTRACT

A method for the treatment or amelioration of Acute Respiratory Distress Syndrome (ARDS) comprising the steps of administering to a subject a therapeutically effective amount of at least one chemokine receptor 2 (CCR2) pathway inhibitor or a pharmaceutically acceptable salt thereof.

### TECHNICAL FIELD

The present invention relates to methods and formulations for the treatment, amelioration and prevention of infection induced acute respiratory distress syndrome, comprising at least one chemokine receptor 2 pathway inhibitor.

### BACKGROUND ART

Acute respiratory distress syndrome (ARDS) is a type of respiratory failure characterized by rapid onset of widespread inflammation in the lungs. The underlying mechanism involves diffuse injury to cells which form the barrier of the microscopic air sacs of the lungs, surfactant dysfunction, activation of the immune system, and dysfunction of the body's regulation of blood clotting. The most common cause of ARDS is sepsis, which may be associated with pneumonia, (bacterial, viral, or fungal) or with a non-pulmonary infectious source, such as peritonitis. Other triggers include inhalation of harmful substances; breathing high concentrations of smoke or chemical fumes; inhaling (aspirating) vomit; and near-drowning episodes.

Coronavirus disease 2019 (COVID-19) is an infectious disease caused by severe acute respiratory syndrome coronavirus 2 (SARS-CoV-2). The World Health Organization (WHO) declared the 2019-20 coronavirus outbreak a pandemic in March 2020. People are managed with supportive care, which may include fluid therapy, oxygen support, and supporting other affected vital organs. However, there is currently no specific, approved treatment for the virus. Many of those infected with the SARS-CoV-2 virus develop ARDS.

There is therefore a need to provide new treatments for ARDS, including ARDS associated with COVID-19; or at least the provision of alternative treatments to compliment the previously known ARDS treatments.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

The present invention provides a method for the treatment, amelioration or prevention of ARDS, said method comprising the step of:
i) administering to a subject a therapeutically effective amount of a chemokine receptor 2 (CCR2) pathway inhibitor.

The present invention also provides a method for the treatment, amelioration or prevention of ARDS, said method comprising the step of:
i) administering to a subject a therapeutically effective amount of (a) an angiotensin type 1 receptor (AT₁R) blocker and (b) a chemokine receptor 2 (CCR2) pathway inhibitor.

Preferably the ARDS is infection related ARDS. For example, the ARDS may be associated with infection by severe acute respiratory syndrome coronavirus 2 (SARS-CoV-2).

Preferably the CCR2 pathway inhibitor is propagermanium and/or the AT₁R blocker is chosen from the group comprising: irbesartan, eprosartan, losartan, valsartan, telmisartan, candesartan, olmesartan, and ZD-7115.

CCR2 pathway inhibitors include pharmaceutically acceptable salts of CCR2 pathway inhibitors. AT₁R blockers include pharmaceutically acceptable salts of AT₁R blockers.

The CCR2 pathway inhibitor and the AT₁R blocker may be administered in the same dosage form or in separate dosage forms.

The CCR2 pathway inhibitor and the AT₁R blocker may be administered concurrently or sequentially.

The invention also provides for the use of at least one chemokine receptor 2 (CCR2) pathway inhibitor for the manufacture of a dosage form for the treatment or prevention of ARDS.

The invention also provides for the use of (a) at least one angiotensin type 1 receptor (AT₁R) blocker, and (b) at least one chemokine receptor 2 (CCR2) pathway inhibitor for the manufacture of a dosage form for the treatment or prevention of ARDS.

The present invention provides a kit for the treatment, amelioration or prevention of ARDS, said kit comprising:

a) at least one chemokine receptor 2 (CCR2) pathway inhibitor; and

b) instructions for use.

The present invention provides a kit for the treatment, amelioration or prevention of ARDS, said kit comprising:

a) at least one angiotensin type 1 receptor (AT₁R) blocker;

b) at least one chemokine receptor 2 (CCR2) pathway inhibitor; and

c) instructions for use.

The present invention provides at least one CCR2 pathway inhibitor, for use in a formulation for the treatment, amelioration or prevention of ARDS.

The present invention provides at least one AT₁R blocker, and at least one CCR2 pathway inhibitor, for use in a formulation for the treatment, amelioration or prevention of ARDS.

The present invention provides at least one AT₁R blocker for use in a formulation for the treatment, amelioration or prevention of ARDS, wherein the at least one AT₁R blocker is administered to the subject concurrently or sequentially with at least one CCR2 pathway inhibitor.

The present invention provides at least one CCR2 pathway inhibitor for use in a formulation for the treatment, amelioration or prevention of ARDS wherein the at least one CCR2 pathway inhibitor is administered to the subject concurrently or sequentially with at least one AT₁R blocker.

### DESCRIPTION OF INVENTION

### Detailed Description of the Invention

### RAAS Dysregulation

The Renin-Angiotensin-Aldosterone System (RAAS) consists of an enzymatic cascade beginning with liver-mediated production of angiotensinogen (AGT), the precursor of angiotensin (Ang) peptides. Ang II, the main effector of this system, results from successive enzymatic actions of renin and angiotensin-converting enzyme (ACE) and exerts most of its actions through activation of Ang II type 1 and type 2 receptors (AT₁R and AT₂R, respectively). In general, AT₁R mediates the pathogenic actions of Ang II, whereas, activated AT₂R elicits protective effects. In the last few decades, novel components of RAAS including (pro)renin receptor, angiotensin-converting enzyme 2 (ACE2), other Ang peptides, and their receptors have been discovered.

The inventors have identified a range of separate and diverse indicators that dysregulation of elements of RAAS is associated with ARDS caused by an infection. ARDS is characterized by rapid onset of widespread inflammation in the lungs, and the RAAS system is associated with inflammation. For example, the infection may be an infection by SARS-CoV-2.

### MCP-1

The inventors have recognized that MCP-1, the ligand for chemokine receptor 2 and an element of the RAAS system, is linked to lung inflammation induced by lipopolysaccharide (LPS) or lipoteichoic acid (LTA), constituents of the Gram-negative and Gram-positive bacterial cell wall, respectively. MCP-1 is a proinflammatory mediator during pulmonary inflammation induced by either LPS or LTA (van Zoelen et al., 2011).

### ACE Inhibitors and AT₁R Inhibitors

The inventors have identified that ACE/Ang-II expression is markedly increased in patients with ARDS and patients with sepsis, the most common cause of ARDS. Clinical epidemiological studies have shown a significant association between polymorphisms in the ACE gene and the susceptibility to ARDS (Wang et al., 2019).

### Novel Use of CCR2 Pathway Inhibitors

Chemokine receptor 2 (CCR2) is another element of the RAAS system. CCR2 inhibition is central in control of a range of inflammatory diseases, although to date CCR2 inhibition programs have only been associated with chronic (not acute) inflammatory diseases such as renal disease. In models of inflammatory renal disease (Ayoub et al., 2015), the inventors have shown that there is a decreased infiltration of monocytes to the damaged kidney, and decreased fibrosis when treated with an AT₁R blocker and a CCR2 pathway inhibitor. In cells such as activated monocytes, where chemotaxis to areas of inflammation is driven by a cline in MCP-1 (also known as CCL2), inhibition of CCR2 alone or in addition to AT₁R, inhibits this chemotaxis.

However, the inventors have recognized the broader application of such CCR2 inhibition programs to acute inflammatory conditions such as ARDS caused by an infection. For example, the infection may be an infection by SARS-CoV-2.

It has been shown that AT₁R and CCR2 functionally interact on the cell surface (Ayoub et al, 2015). When both receptors and their ligands are present, it is beneficial to simultaneously inhibit both receptors, with a synergistic effect in reducing endpoints of inflammation. Optimal inhibition of CCR2 is achieved when AT₁R is simultaneously blocked.

Thus, regulation of the RAAS pathway in subjects with ARDS may be achieved by administration of (i) a CCR2 pathway inhibitor, or (ii) a CCR2 pathway inhibitor and an AT₁R blocker.

### Composition

The present invention provides a pharmaceutical formulation comprising:

a) at least one chemokine receptor 2 (CCR2) pathway inhibitor.

The present invention provides a pharmaceutical formulation comprising:

a) at least one angiotensin type 1 receptor (AT₁R) blocker; and

b) at least one chemokine receptor 2 (CCR2) pathway inhibitor.

The pharmaceutical formulation may optionally include excipients, solvents, carriers and other pharmaceutically acceptable ingredients.

The term “component” as used herein in the context of a pharmaceutical formulation of the invention, means either the AT₁R blocker or the CCR2 pathway inhibitor.

### CCR2 Inhibitor

Chemokine receptor 2 (CCR2) is a G-protein coupled receptor.

The phrase “chemokine receptor 2 pathway” is to be understood to include any one of the pathways of the chemokine receptor 2 activated by its native ligands, including but not limited to those pathways that cause chemotactic migration, cell motility, extracellular-regulated kinase (ERK) phosphorylation, cAMP production, actin-recruitment, protein phosphorylation, nuclear protein localization, gene transcription or translation, G protein coupling, β-arrestin recruitment or mediated signalling.

The phrase “chemokine receptor 2 pathway inhibitor” is intended to include any compound or agent which inhibits or partially inhibits any one of the pathways associated with the chemokine receptor 2. The chemokine receptor pathway inhibitor may be a peptide, polypeptide or small chemical entity. For example, the chemokine receptor 2 pathway inhibitor may be a protein, binding protein or antibody.

The phrase “chemokine receptor 2 pathway inhibitor” includes compounds or agents which inhibit or partially inhibit the chemokine receptor 2 itself. Preferably, such chemokine receptor 2 pathway inhibitors are selected from: a direct CCR2 antagonist, a direct CCR2 inverse agonist or a direct CCR2 negative allosteric modulator.

The phrase “chemokine receptor 2 pathway inhibitor” includes compounds or agents which inhibit or partially inhibit a component of the chemokine receptor pathway other than the chemokine receptor 2 itself. For example, the inhibitor may inhibit or partially inhibit proteins that associate with chemokine receptor 2 or may inhibit compounds or pathway steps before and/or after the chemokine receptor itself. In such a case, preferably, the chemokine receptor 2 pathway inhibitors are selected from: an indirect CCR2 antagonist, an indirect CCR2 inverse agonist or an indirect CCR2 negative allosteric modulator.

The term “a component of the chemokine receptor pathway other than the chemokine receptor 2 itself” as used herein, is to be understood to refer a component of the chemokine receptor 2 pathway wherein the component is itself not the chemokine receptor 2. The component of the chemokine receptor pathway may interact directly with chemokine receptor 2. Alternatively, the component of the chemokine receptor pathway may interact indirectly with chemokine receptor 2 by way of protein-protein interaction or complex formation. Alternatively, the component of the chemokine receptor pathway may interact indirectly with chemokine receptor 2 by way of a signalling cascade. Preferably, the component is a protein such as, but not limited to, a transduction or signalling protein.

In one aspect, if the chemokine receptor pathway inhibitor inhibits or partially inhibits a component of the pathway other than the chemokine receptor 2 itself, the inhibitor blocks MCP-1 induced migration and activation of monocytes and chemotactic migration through the targeting of glycosylphosphatidylinositol (GPI)-anchored proteins such as CD55, CD59 and CD16.

In one aspect, if the chemokine receptor pathway inhibitor inhibits or partially inhibits a component of the pathway other than the chemokine receptor 2, the inhibited component is MCP-1. In one embodiment the chemokine receptor 2 pathway inhibitor inhibits or partially inhibits the in vitro chemotactic migration of monocytes induced by MCP-1 by targeting MCP-1 directly. MCP-1 is a ligand for chemokine receptor 2. Such compounds include agents which bind MCP-1.

In another aspect, if the chemokine receptor pathway inhibitor inhibits or partially inhibits a component of the pathway other than the chemokine receptor 2, the inhibited component is chosen from the list of CCR2 ligands comprising: CCL7 (MCP-3), CCL8, CCL13 (MCP-4), and CCL16 [hemofiltrate CC chemokine (HCC)-4]. In one embodiment the chemokine receptor 2 pathway inhibitor inhibits or partially inhibits the in vitro chemotactic migration of monocytes induced by the above CCR2 ligands by targeting the ligands directly. Such compounds include agents which bind CCL7, CCL8, CCL13, and CCL16.

In another embodiment the chemokine receptor 2 pathway inhibitor inhibits or partially inhibits the in vitro chemotactic migration of monocytes induced by MCP-1 by targeting one or more GPI-anchored proteins selected from the group comprising CD55, CD59 and CD16. In such as case, the chemokine receptor pathway inhibitor may stabilize the complexes CCR2/CD55 and/or CCR2/CD59 and/or CCR2/CD16.

Known inhibitors of the chemokine receptor 2 pathway include: propagermanium or repagermanium; RS504393; RS102895: MLN-1202 (Millennium Pharmaceuticals); INCB3344, INCB3284 and INCB8696 (Incyte Pharmaceuticals); MK-0812 (Merck); CCX140 (ChemoCentryx); PF-4136309 (Pfizer); BMS-741672 (Bristol-Myers Squibb).

In one preferred embodiment the chemokine receptor pathway inhibitor is a direct antagonist of chemokine receptor 2, such as propagermanium, repagermanium or CCX140.

Propagermanium (3-oxygermylpropionic acid polymer), is a molecule that has been used as a therapeutic agent against chronic hepatitis, also has been shown to specifically inhibit in vitro chemotactic migration of monocytes by MCP-1 through a mechanism that seems to require glycosylphosphatidylinositol (GPI)-anchored proteins such as CD55, CD59 and CD16 (Yokochi, S. 2001). Propagermanium is also known as 3-[(2-Carboxyethyl-oxogermyl)oxy-oxogermyl]propanoic acid, proxigermanium, Ge-132, bis (2-carboxyethylgermanium) sesquioxide (CEGS), 2-carboxyethylgermasesquioxane, SK-818, organic germanium, germanium sesquioxide, 3,3<′>-(1,3-dioxo-1,3-digermanoxanediyl) bispropionic acid, 3-oxygermylpropionic acid polymer, poly-fra/7s-(2-carboxyethyl) germasesquioxane, proxigermanium, repagermanium and Serocion™.

Whilst propagermanium has not been tested in the respiratory setting, it has an extensive safety database with a low adverse event profile unlike many immune modulators. Propagermanium is approved in Japan, having been registered in 1994 as an immune modulator for chronic hepatitis B treatment. Propagermanium has also been in use in clinical practice for renal patients under the TGA Special Access Scheme since 2017.

### Angiotensin Type 1 Receptor

The angiotensin type 1 receptor (AT₁R, AT1R, angiotensin II receptor type 1) is a G protein-coupled receptor.

The phrase “angiotensin type 1 receptor blocker” (also referred to as AT₁R blocker or ARB) is understood to include any compound or agent which inhibits or partially inhibits the activation of AT₁R.

This includes AT₁R antagonists, AT₁R inverse agonists and AT₁R negative allosteric modulators.

For example, the AT₁R blocker may be selected from the group comprising: irbesartan (e.g. Avapro®), eprosartan (e.g. Teveten®), losartan (e.g. Cozaar®), valsartan (e.g. Diovan®), telmisartan (e.g. Micardis®), candesartan (e.g. Atacand®), olmesartan (e.g. Benicar®), Azilsartan (Edarbi) and ZD-7115.

Taylor et al (2011) reviews AT₁R blockers and noted that “[t]here are no major differences in efficacy or other clinical characteristics among older drugs in this class, although some of the newer agents may more effectively reduce blood pressure than the older agents”. The differences in characteristics appear to be related to interaction with other pathways and not the core angiotensin type 1 receptor blockage. It is noted that although “[t]here are some pharmacological differences among the ARBs, and some drugs in this class have special effects independent of angiotensin II receptor blockade, yet there is currently no strong evidence that these translate into unique protection against target organ damage from hypertension”. Thus, in respect of their effect on AT₁R blockage, AT₁R blockers are functionally interchangeable despite structural differences.

ARBs first become commercially available in the 1990's. Despite the extended period of use of this class of drugs, physicians treat these as a group which are largely substitutable with each other. While there are some specific subtleties in the exact pharmacology of some of the group, these appear not to be related to their angiotensin type 1 receptor activity. Furthermore, all pharmacological products will behave subtly differently in some patients, and yet some sub-group variability does not prevent them from being generally useful in therapy (Taylor et al., 2011).

As an example, the angiotensin receptor inhibitor may be irbesartan. Irbesartan is an angiotensin type 1 receptor antagonist also known as 2-butyl-3-({4-[2-(2H-1,2,3,4-tetrazol-5-yl)phenyl]phenyl}methyl)-1,3-diazaspiro[4.4]non-1-en-4-one.

Both the AT₁R blocker and the CCR2 pathway inhibitor may be pharmaceutically acceptable salts of the respective active agent. Pharmaceutically and veterinary acceptable salts include salts which retain the biological effectiveness and properties of the compounds of the present disclosure and which are not biologically or otherwise undesirable. In many cases, the compounds disclosed herein are capable of forming acid and/or base salts by virtue of the presence of amino and/or carboxyl groups or groups similar thereto. Acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as by way of example only, alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amines, trisubstituted cycloalkyl amines, cycloalkenyl amines, di(cycloalkenyl) amines, tri(cycloalkenyl) amines, substituted cycloalkenyl amines, disubstituted cycloalkenyl amines, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Pharmaceutically and veterinary acceptable acid addition salts may be prepared from inorganic and organic acids. The inorganic acids that can be used include, by way of example only, hydrochloric acid, hydrobromic acid, sulfuric acid, nitric acid, phosphoric acid, and the like. The organic acids that can be used include, by way of example only, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The pharmaceutically or veterinary acceptable salts of the compounds useful in the present disclosure can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences. 17th ed., Mack Publishing Company, Easton, Pa. (1985), p. 1418, the disclosure of which is hereby incorporated by reference. Examples of such acceptable salts are the iodide, acetate, phenyl acetate, trifluoroacetate, acrylate, ascorbate, benzoate, chlorobenzoate, dinitrobenzoate, hydroxybenzoate, methoxybεnzoate, methylbenzoate, o-acetoxybenzoate, naphthalene-2-benzoate, bromide, isobutyrate, phenylbutyrate, γ-hydroxybutyrate, 3-hydroxybutyrate, butyne-1,4-dioate, hexyne-1,4-dioate, hexyne-1,6-dioate, caproate, caprylate, chloride, cinnamate, citrate, decanoate, formate, fumarate, glycollate, heptanoate, hippurate, lactate, malate, maleate, hydroxymaleate, malonate, mandelate, mesylate, nicotinate, isonicotinate, nitrate, oxalate, phthalate, terephthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzenesulfonate, p-bromophenylsulfonate, chlorobenzenesulfonate, propanesulfonate, ethanesulfonate, 2-hydroxyethanesulfonate, merhanesulfonate, naphthalene-1-sulfonate, naphthalene-2-sulfonate, p-toluenesulfonate, xylenesulfonate, tartarate, and the like.

### Condition or Disease

Preferably the CCR2 pathway inhibitor and/or the AT₁R blocker are for use in the treatment, amelioration or prevention of ARDS. ARDS is a type of respiratory failure characterized by rapid onset of widespread inflammation in the lungs.

The most common cause of ARDS is primary pneumonia, which can be bacterial, viral, or fungal. The second most common cause of lung injury is severe sepsis, which may be associated with pneumonia or a non-pulmonary infectious source, such as peritonitis. The other important major causes of ARDS include aspiration of gastric contents; haemorrhage and shock following major trauma; and several other less common causes such as severe acute pancreatitis, transfusion-associated lung injury, and drug reactions.

Recent experience has also found that ARDS may be found in subjects with infection by severe acute respiratory syndrome coronavirus 2 (SARS-CoV-2). Thus, the ARDS may be associated with infection by SARS-CoV-2.

The term “prevention” includes administration of the compositions of the present invention to subjects who have symptoms that indicate they are at risk of developing ARDS, but may not, at the time of treatment, be diagnosed with ARDS. For example, the subject may have severe persistent cough and/or low oxygen levels, but not be diagnosed with ARDS.

The term “amelioration” includes administration of the compositions of the present invention to subjects who have had ARDS and are at risk of developing post-ARDS conditions such as fibrosis and persistent inflammation. The post-ARDS conditions may be due to the ARDS itself or may be due to the use of ventilators during the acute phase of ARDS.

### Measurement of Inhibition

The phrase “angiotensin type 1 receptor blocker” is understood to include any compound or agent which inhibits or partially inhibits the activation of AT₁R.

The phrase “chemokine receptor 2 pathway inhibitor” is intended to include any compound or agent which inhibits or partially inhibits any one of the pathways associated with the chemokine receptor 2.

The term “inhibits” as used herein, means a reduction below detectable limits when compared to a reference. The phrase includes blocking, retarding, or impeding an action to prevent an undesirable result.

The term “partially inhibits” as used herein, means any reduction within detectable limits when compared to a reference. The phrase includes blocking, retarding, or impeding an action to prevent an undesirable result.

The inhibition or partial inhibition of the CCR2 pathway and/or the AT₁R caused by (i) the CCR2 pathway inhibitor, (ii) the AT₁R blocker, or (iii) both the CCR2 pathway inhibitor and the AT₁R blocker, may be measured using the in vitro methods set out herein, and include but are not limited to, biochemical or cellular assays for the assessment of in vitro chemotactic migration of CCR2-expressing neutrophils and other cells such as are known in the art, as well as measurement of inositol phosphate production, extracellular-regulated kinase (ERK) phosphorylation, cAMP production, actin-recruitment, protein phosphorylation, nuclear protein localization, gene transcription, label-free technologies (such as using impedance, light refraction or charge redistribution), G protein coupling using proximity reporter systems or other approaches, β-arrestin recruitment or mediated signalling, transcription factor-based reporter systems, microscopy visualization using fluorescent labels, use of antibodies to assess receptor cellular localization (such as enzyme-linked immunosorbent assays) and fluorescence activated cell sorting.

The inhibition or partial inhibition of the CCR2 pathway and/or the AT₁R caused by (i) the CCR2 pathway inhibitor, (ii) the AT₁R blocker, or (iii) both the CCR2 pathway inhibitor and the AT₁R blocker, may be measured using the in vivo methods set out herein, and include but are not limited to, measurement of cellular and cytokine content of lung exudate, measurement of lung function including physical capacity of lung function using spirometry-based tests, or lung functional outputs measured using measurement blood gas or other biochemical measures, or improvement in functional benefit including clinical benefit measured by survival or quantitative methods such as walk tests or qualitative methods such as patient-reported outcome assessment. Inhibition or partial inhibition may be indicated by a qualitative improvement in lung structure as measured by one or more of the above-mentioned endpoints.

In another embodiment, the total efficacy of the pharmaceutical formulation is greater when compared to the efficacies of the AT₁R blocker or the CCR2 pathway inhibitor when either component is administered without any administration of the other component. Thus, the combined formulation may be administered in a single dose, including at sub-therapeutic doses, or less often, than either of the two components might be administered as single compounds.

Preferably, the total efficacy of the pharmaceutical formulation is greater when compared to the sum of the efficacies of the AT₁R blocker and the CCR2 pathway inhibitor when either component is administered without any administration of the other component. More preferably, a synergistic effect in efficacy is observed when the AT₁R blocker and the CCR2 pathway inhibitor are administered concurrently or sequentially.

Alternatively, the total efficacy of the pharmaceutical formulation is equal to the sum of the efficacies of the AT₁R blocker and the CCR2 pathway inhibitor when either component is administered without any administration of the other component. As a further preferred embodiment of this alternative, an additive effect in efficacy is observed when the AT₁R blocker and the CCR2 pathway inhibitor are administered concurrently or sequentially.

In a further alternative, the total efficacy of the pharmaceutical formulation is less than the sum of the efficacies of the AT₁R blocker and the CCR2 pathway inhibitor when either component is administered without any administration of the other component. In a further embodiment, while the combined efficacy is less than the sum of the efficacies of the AT₁R blocker and the CCR2 pathway inhibitor when each component is administered without any administration of the other component, the treatment provides greater efficacy compared to a single treatment of AT₁R blocker or the CCR2 pathway inhibitor administered alone.

Preferably the two components are administered concurrently at the same time (for example as two tablets taken together, or as a single tablet, formulated with each component) or sequentially (for example one tablet taken after another tablet). The doses of each component may be taken together (concurrently), or sequentially and taken within seconds, minutes, days, weeks or months of each other.

### Method of Treatment

The invention provides a method for treatment, amelioration or prevention of ARDS, said method comprising the step of:
i) administering to a subject a therapeutically effective amount of a chemokine receptor 2 (CCR2) pathway inhibitor.

The invention further provides a method for treatment, amelioration or prevention of ARDS, said method comprising the step of:
i) administering to a subject a therapeutically effective amount of (a) an angiotensin type 1 receptor (AT₁R) blocker, and (b) a chemokine receptor 2 (CCR2) pathway inhibitor.

The subject to be treated is preferably a mammal, including a human mammal.

Preferably the ARDS is infection related ARDS. For example, the ARDS may be associated with infection by severe acute respiratory syndrome coronavirus 2 (SARS-CoV-2).

The CCR2 pathway inhibitor may be administered alone, as a single therapy.

Alternatively, the CCR2 pathway inhibitor and the AT₁R blocker may be administered: in the same dosage form or in separate dosage forms. The CCR2 pathway inhibitor and the AT₁R blocker may be administered: concurrently or sequentially.

The CCR2 pathway inhibitor and/or the AT₁R blocker may be pharmaceutically acceptable salts of the CCR2 pathway inhibitor and/or the AT₁R blocker.

One component of the treatment of the present invention may already be being administered to a subject, for example as standard of care treatment. In such a case, the second component of the treatment of the present invention is administered as a second component in therapy to provide the therapy of the present invention.

While not intending to be restricted to any particular mode of action, in one preferred embodiment the CCR2 pathway inhibitor has a greater affinity and/or potency and/or efficacy when interacting with the CCR2 or modulating its downstream pathways when the CCR2 is associated with the angiotensin receptor. For example, the CCR2 and the angiotensin receptor may be associated as a CCR2/AT₁R hetero-dimer/-oligomer. In a further preferred embodiment, when the CCR2 pathway inhibitor is administered to a subject concurrently or sequentially with an AT₁R blocker, the combined affinity, potency and/or efficacy is greater than compared to the affinity, potency and/or efficacy that would have been achieved when the CCR2 pathway inhibitor is not administered (whether concurrently or sequentially) with the AT₁R blocker. In an even further preferred embodiment, a synergistic effect (as measured by affinity, potency and/or efficacy) is achieved when the CCR2 pathway inhibitor is administered to a subject (whether concurrently or sequentially) with an AT₁R blocker.

While not intending to be restricted to any particular mode of action, in one preferred embodiment the AT₁R blocker has a greater affinity and/or potency and/or efficacy when interacting with the angiotensin receptor when the angiotensin receptor is associated with the CCR2. For example, the CCR2 and the angiotensin receptor may be associated as a CCR2/AT₁R hetero-dimer/-oligomer. In a further preferred embodiment, when the AT₁R blocker is administered to a subject concurrently or sequentially with a CCR2 pathway inhibitor, the combined affinity, potency and/or efficacy is greater than compared to the affinity, potency and/or efficacy that would have been achieved when the AT₁R blocker is not administered (whether concurrently or sequentially) with the CCR2 pathway inhibitor. In an even further preferred embodiment, a synergistic effect (as measured by affinity, potency and/or efficacy) is achieved when the AT₁R blocker is administered to a subject (whether concurrently or sequentially) with a CCR2 pathway inhibitor.

### Delivery

The dosage form provided by the present invention may further comprise a vial, cartridge, container, tablet or capsule comprising the pharmaceutical formulation of the invention together with dosage instructions for the administration of the dosage form(s) to a subject for the treatment, amelioration or prevention of ARDS.

The amount of each active ingredient which may be combined with the carrier materials to produce a single dosage will vary, depending upon the host to be treated and the particular mode of administration. For example, a formulation intended for oral administration to humans may contain about 0.5 mg to 1 g of each active compound with an appropriate and convenient amount of carrier material, which may vary from about 5 to 95% w/w of the total formulation. Dosage unit forms will generally contain between from about 0.5 mg to 1000 mg of active ingredient(s).

Preferably, the CCR2 pathway inhibitor is provided at between 0.5 mg to 2000 mg per day, provided in one or more doses. Even more preferably the CCR2 pathway inhibitor is provided at a dose of between 0.5 mg to 50 mg per day, provided in one or more doses.

The AT₁R blocker may be provided at between 50 mg to 500 mg per day, provided in one or more doses. The AT₁R blocker may be provided at between 75 mg to 300 mg per day. For example, the AT₁R blocker may be irbesartan and is administered at a dose of 75, 150 or 300 mg per day, provided in one or more doses.

Preferably, the AT₁R blocker is provided at between 1 mg to 1200 mg per day, provided in one or more doses. In one form of the invention:
the AT₁R blocker is irbesartan and it is administered in an individual dose of 75, 150 or 300 mg, provided one or more times per day;
the AT₁R blocker is eprosartan mesylate and it is administered in an individual dose of 300 mg, 400 mg or 600 mg, provided one or more times per day;
the AT₁R blocker is losartan potassium and it is administered in an individual dose of 25 mg, 50 mg or 100 mg, provided one or more times per day;
the AT₁R blocker is valsartan and it is administered in an individual dose of 80 mg, 160 mg or 320 mg, provided one or more times per day;
the AT₁R blocker is telmisartan and it is administered in an individual dose of 20 mg, 40 mg or 80 mg, provided one or more times per day;
the AT₁R blocker is candesartan cilexetil and it is administered in an individual dose of 4 mg, 8 mg, 16 mg or 32 mg, provided one or more times per day;
the AT₁R blocker is olmesartan medoxomil and it is administered in an individual dose of 5 mg, 10 mg, 20 mg or 40 mg, provided one or more times per day;
the AT₁R blocker is azilsartan kamedoxomil and it is administered in an individual dose of 40 mg or 80 mg, provided one or more times per day.

By individual dose, it is meant that the required amount of AT₁R blocker is delivered in a dosage form.

If the AT₁R blocker is not the prodrug form or salt listed above, the dose is the equivalent of the amount of active compound.

The dose of each active agent may be provided in either a single dosage form, or two separate dosage forms. The actives may be provided as about 0.5 mg to 1 g of the CCR2 pathway inhibitor and about 1 mg to 1200 mg of the AT₁R blocker. The dose of the two actives may be provided in either a single dosage form, or two separate dosage forms and may comprise (i) a daily dose of AT₁R blocker of between about 1 mg to 1200 mg, and (ii) a daily dose of CCR2 pathway inhibitor of between about 5 mg to 50 mg. The AT₁R blocker may be irbesartan, and the dosage form may comprise a daily dose of irbesartan of about 300 mg.

TABLE 1
Examples of combinations of preferred active agents:

| | |
|---|---|
| COMBINATION 1A | Repagermanium 120 mg |
| | Candesartan 4 mg |
| COMBINATION 1B | Repagermanium 120 mg |
| | Candesartan 8 mg |
| COMBINATION 1C | Repagermanium 120 mg |
| | Candesartan 16 mg |
| COMBINATION 1D | Repagermanium 120 mg |
| | Candesartan 32 mg |
| COMBINATION 2A | Repagermanium 120 mg |
| | Irbesartan 75 mg |
| COMBINATION 2B | Repagermanium 120 mg |
| | Irbesartan 150 mg |
| COMBINATION 2C | Repagermanium 120 mg |
| | Irbesartan 300 mg |
| COMBINATION 3A | Repagermanium 120 mg |
| | Eprosartan 300 mg |
| COMBINATION 3B | Repagermanium 120 mg |
| | Eprosartan 400 mg |
| COMBINATION 3B | Repagermanium 120 mg |
| | Eprosartan 600 mg |
| COMBINATION 4A | Repagermanium 120 mg |
| | Losartan 25 mg |
| COMBINATION 4B | Repagermanium 120 mg |
| | Losartan 50 mg |
| COMBINATION 4C | Repagermanium 120 mg |
| | Losartan 100 mg |
| COMBINATION 5A | Repagermanium 120 mg |
| | Valsartan 40 mg |
| COMBINATION 5B | Repagermanium 120 mg |
| | Valsartan 80 mg |
| COMBINATION 5C | Repagermanium 120 mg |
| | Valsartan 160 mg |
| COMBINATION 5D | Repagermanium 120 mg |
| | Valsartan 320 mg |
| COMBINATION 6A | Repagermanium 120 mg |
| | Telmisartan 20 mg |
| COMBINATION 6B | Repagermanium 120 mg |
| | Telmisartan 40 mg |
| COMBINATION 6C | Repagermanium 120 mg |
| | Telmisartan 80 mg |
| COMBINATION 7A | Repagermanium 120 mg |
| | Olmesartan 5 mg |
| COMBINATION 7B | Repagermanium 120 mg |
| | Olmesartan 10 mg |
| COMBINATION 7C | Repagermanium 120 mg |
| | Olmesartan 20 mg |
| COMBINATION 7D | Repagermanium 120 mg |
| | Olmesartan 40 mg |
| COMBINATION 8 | Repagermanium |
| | ZD-7115 |
| COMBINATION 9A | Repagermanium 120 mg |
| | Sparsentan 200 mg |
| COMBINATION 9B | Repagermanium 120 mg |
| | Sparsentan 400 mg |
| COMBINATION 9C | Repagermanium 120 mg |
| | Sparsentan 800 mg |
| COMBINATION 10A | Repagermanium 120 mg |
| | Azilsartan 40 mg |
| COMBINATION 10B | Repagermanium 120 mg |
| | Azilsartan 80 mg |

In one embodiment the repagermanium and the AT₁R blocker are provided as once daily doses. In another embodiment the repagermanium and the AT₁R blocker are provided as twice daily doses. In a preferred embodiment the repagermanium is provided as a twice daily dose (BID) and the AT₁R blocker is administered as a single daily dose.

It will be understood, however, that the specific dose level for any particular subject will depend upon a variety of factors including the activity of the specific compound employed, the age, body weight, general health, sex, diet, time of administration, route of administration, rate of excretion, combinations of drugs, and the severity of the particular condition or disease undergoing therapy.

Formulations of the invention, in various aspects, may be administered by injection, or prepared for oral, pulmonary, nasal or for any other form of administration. Preferably the formulations are administered, for example, intravenously, subcutaneously, intramuscularly, intraorbitally, ophthalmically, intraventricularly, intracranially, intracapsularly, intraspinally, intracisternally, intraperitoneally, buccal, rectally, vaginally, intranasally or by aerosol administration.

The mode of administration is in one aspect at least suitable for the form in which the formulation has been prepared. The mode of administration for the most effective response may be determined empirically and the means of administration described below are given as examples, and do not limit the method of delivery of the formulation of the present invention in any way. All the formulations provided are commonly used in the pharmaceutical industry and are commonly known to suitably qualified practitioners.

### Injectable Dosage Forms

The formulations of the invention in certain aspects may include pharmaceutically acceptable non-toxic excipients and carriers and administered by any parenteral techniques such as subcutaneous, intravenous and intraperitoneal injections. In addition, the formulations may optionally contain one or more adjuvants. As used herein, a “pharmaceutical carrier” is a pharmaceutically acceptable solvent, suspending agent, excipient or vehicle for delivering the compounds to the subject. The carrier may be liquid or solid and is selected with the planned manner of administration in mind.

The pharmaceutical forms suitable for injectable use optionally include sterile aqueous solutions (where water-soluble) or dispersions and sterile powders for the extemporaneous preparation of sterile injectable solutions or dispersion. Alternatively, the compounds of the invention are, in certain aspects encapsulated in liposomes and delivered in injectable solutions to assist their transport across cell membrane. Alternatively, or in addition, such preparations contain constituents of self-assembling pore structures to facilitate transport across the cellular membrane. The carrier, in various aspects, is a solvent or dispersion medium containing, for example, water, ethanol, polyol (for example, glycerol, propylene glycol and liquid polyethylene glycol, and the like), suitable mixtures thereof, and vegetable oils. Proper fluidity is maintained, for example and without limitation, by a coating such as lecithin; by the maintenance of the required particle size in the case of dispersion; and by the use of surfactants.

The invention also provides prolonged absorption of an injectable sustained release pharmaceutical formulation comprising a therapeutically effective pharmaceutical formulation according to the invention and a release retardant. The release retardant may be, for example, aluminium mono-stearate and gelatine.

Sterile injectable solutions are prepared by incorporating the active compounds in the required amount in an appropriate solvent with one or more of the other ingredients enumerated above, as required, followed by filtered sterilisation. Generally, dispersions are prepared by incorporating the various sterilised active ingredient into a sterile vehicle that contains the basic dispersion medium and the required other ingredients from those enumerated above. In the case of sterile powders for the preparation of sterile injectable solutions, preparation in certain aspects include without limitation vacuum drying and freeze-drying techniques that yield a powder of the active ingredient plus any additional desired ingredient from previously sterile-filtered solution thereof.

### Oral Dosage Forms

Contemplated for use herein are oral dosage forms, which are described generally in Martin, Remington's Pharmaceutical Sciences, 18th Ed. (1990 Mack Publishing Co. Easton Pa. 18042) at Chapter 89, which is herein incorporated by reference. Solid dosage forms include tablets, capsules, pills, troches or lozenges, cachets, liquids, suspensions or pellets. Also, liposomal or proteinoid encapsulation may be used to formulate the present formulations (as, for example, proteinoid microspheres reported in U.S. Pat. No. 4,925,673). Liposomal encapsulation may be used, and the liposomes may be derivatised with various polymers (E.g., U.S. Pat. No. 5,013,556). A description of possible solid dosage forms for the therapeutic is given by Marshall, in Modern Pharmaceutics, Chapter 10, Banker and Rhodes ed., (1979), herein incorporated by reference. In general, the formulation will include the compounds described as part of the invention (or a chemically modified form thereof), and inert ingredients which allow for protection against the stomach environment, and release of the biologically active material in the intestine.

The oral dosage form of the present invention may be a liquid, suspension or other appropriate dosage form that is delivered directly into the feeding tube of a patient who is in a coma and/or on a respirator and is unable to swallow a tablet etc.

For the CCR2 pathway inhibitor or AT₁R blocker of the invention the location of release may be the stomach, the small intestine (the duodenum, the jejunum, or the ileum), or the large intestine. One skilled in the art has available formulations that will not dissolve in the stomach yet will release the material in the duodenum or elsewhere in the intestine. In one aspect, the release will avoid the deleterious effects of the stomach environment, either by protection of the formulation or by release of the compounds beyond the stomach environment, such as in

A coating or mixture of coatings can also be used on tablets, which are not intended for protection against the stomach. This includes without limitation sugar coatings, or coatings that make the tablet easier to swallow. Exemplary capsules consist of a hard shell (such as gelatine) for delivery of dry therapeutic i.e. powder; for liquid forms, a soft gelatine shell may be used. The shell material of cachets in certain aspects is thick starch or other edible paper. For pills, lozenges, moulded tablets or tablet triturates, moist massing techniques are also contemplated, without limitation.

As used herein, the term “sustained release” means the gradual but continuous or sustained release over a relatively extended period of the therapeutic compound content after oral ingestion. The release may continue after the pharmaceutical formulation has passed from the stomach and through until and after the pharmaceutical formulation reaches the intestine. The phrase “sustained release” also means delayed release wherein release of the therapeutic compound is not immediately initiated upon the pharmaceutical formulation reaching the stomach but rather is delayed for a period of time, for example, until when the pharmaceutical formulation reaches the intestine. Upon reaching the intestine, the increase in pH may then trigger release of the therapeutic compound from the pharmaceutical formulation.

Though term “release retardant” is used herein, means a substance that reduces the rate of release of a therapeutic compound from a pharmaceutical formulation when orally ingested. The release retardant may be a polymer or a non-polymer. The release retardant may be used according to any one of several sustained release systems including, for example, a diffusion system, a dissolution system and/or an osmotic system.

In certain aspects, the therapeutic is included in the formulation as fine multi-particulates in the form of granules or pellets of particle size about 1 mm. The formulation of the material for capsule administration is, in certain aspects, a powder, lightly compressed plugs or even as tablets. In one aspect, the therapeutic could be prepared by compression.

Colourants and flavouring agents are optionally all be included. For example, compounds may be formulated (such as, and without limitation, by liposome or microsphere encapsulation) and then further contained within an edible product, such as a refrigerated beverage containing colorants and flavouring agents.

The volume of the therapeutics, in one aspect, is diluted or increased with an inert material. These diluents could include carbohydrates, especially mannitol, alpha-lactose, anhydrous lactose, lactose monohydrate, cellulose, silicified microcrystalline cellulose, sucrose, modified dextrans and starch. Certain inorganic salts are also optionally used as fillers including calcium triphosphate, magnesium carbonate and sodium chloride. Some commercially available diluents are Fast-Flo, Emdex, STA-Rx 1500, Emcompress and Avicell.

In other embodiments, disintegrants are included in the formulation of the therapeutic into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatine, orange peel, acid carboxymethyl cellulose, natural sponge and bentonite are also contemplated. Another form of the disintegrants is the insoluble cationic exchange resins. Powdered gums are also optionally used as disintegrants and as binders and these include, without limitation, powdered gums such as agar, Karaya or tragacanth. Alginic acid and its sodium salt and crospovidone are also useful as disintegrants.

Binders are contemplated to hold the therapeutic compounds together to form a hard tablet and include, without limitation, materials from natural products such as acacia, tragacanth, starch and gelatine. Other binders include, without limitation, methylcellulose (MC), ethyl cellulose (EC) and carboxymethyl cellulose (CMC) silicified microcrystalline cellulose (SMCC), polyvinyl pyrrolidone (PVP) and hydroxypropylmethyl cellulose (HPMC). Polyvinyl pyrrolidone (PVP) and hydroxypropylmethyl cellulose (HPMC) are also contemplated for use in alcoholic solutions to granulate the therapeutic

An antifrictional agent may be optionally included in the formulation of the therapeutic to prevent sticking during the formulation process. Lubricants may be optionally used as a layer between the therapeutic and the die wall, and these can include but are not limited to: stearic acid including its magnesium and calcium salts, polytetrafluoroethylene (PTFE), liquid paraffin, vegetable oils and waxes. Exemplary soluble lubricants may also be used such as include sodium lauryl sulfate, magnesium lauryl sulfate, sodium stearyl fumarate, polyethylene glycol of various molecular weights, and Carbowax 4000 and 6000.

Glidants that might improve the flow properties of the compound during formulation and to aid rearrangement during compression might be optionally added. The glidants may include without limitation starch, talc, pyrogenic silica and hydrated silicoaluminate.

To aid dissolution of the therapeutic into the aqueous environment, a surfactant might be added in certain embodiments as a wetting agent. Surfactants may include, for example and without limitation, anionic detergents such as sodium lauryl sulfate, dioctyl sodium sulfosuccinate and dioctyl sodium sulfonate. Cationic detergents might be optionally used and could include, without limitation, benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. When used, these surfactants could be present in the formulation of the compounds either alone or as a mixture in different ratios.

Additives that potentially enhance uptake of the compounds are for instance and without limitation the fatty acids oleic acid, linoleic acid and linolenic acid.

Controlled release formulation may be desirable. The formulations are also contemplated. In certain aspects, the compounds could be incorporated into an inert matrix that permits release by either diffusion or leaching mechanisms i.e., gums. In some aspects, slowly degenerating matrices may also be incorporated into the formulation. Another form of a controlled release of this therapeutic is by a method based on the Oros therapeutic system (Alza Corp.), i.e. the drug is enclosed in a semipermeable membrane which allows water to enter and push drug out through a single small opening due to osmotic effects. Some enteric coatings also have a delayed release effect.

In other aspects, a mix of materials might be used to provide the optimum film coating. Film coating may be carried out, for example and without limitation, in a pan coater or in a fluidized bed or by compression coating.

### Pulmonary and Nasal Dosage Forms

Also contemplated herein is pulmonary delivery of the formulations of the invention. In these aspects, the CCR2 pathway inhibitor or the AT₁R blocker may be delivered to the lungs of a subject while inhaling and traverses across the lung epithelial lining to the blood stream.

Contemplated for use in the practice of this invention are a wide range of mechanical devices designed for pulmonary delivery of therapeutic products, including but not limited to nebulizers, metered-dose inhalers, and powder inhalers, all of which are familiar to those skilled in the art.

Some specific examples of commercially available devices suitable for the practice of this invention are, for example and without limitation, the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the compounds. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated.

Formulations suitable for use with a nebulizer, either jet or ultrasonic, will typically comprise the compounds suspended in water. The formulation may also include, in one aspect, a buffer and a simple sugar (e.g., for protein stabilization and regulation of osmotic pressure). In one embodiment, the nebulizer formulation may also contain a surfactant, to reduce or prevent surface induced aggregation of the compounds caused by atomization of the solution in forming the aerosol.

Formulations for use with a metered dose inhaler device will generally comprise, in one aspect a finely divided powder containing the compounds suspended in a propellant with the aid of a surfactant. The propellant may be is any conventional material employed for this purpose, such as and without limitation, a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include, without limitation sorbitan trioleate and soya lecithin. Oleic acid may also be useful as a surfactant in certain aspects.

Formulations for dispensing from a powder inhaler device will comprise a finely divided dry powder containing the compound and may also include a bulking agent, such as and without limitation lactose, sorbitol, sucrose, or mannitol in amounts which facilitate dispersal of the powder from the device, e.g., 50 to 90% by weight of the formulation. In certain embodiments, the compound(s) is/are prepared in particulate form with an average particle size of less than 10 microns, most preferably 0.5 to 5 microns, for most effective delivery to the distal lung.

Nasal delivery of the compounds is also contemplated. Nasal delivery allows the passage of the protein to the blood stream directly after administering the therapeutic product to the nose, without the necessity for deposition of the product in the lung. Formulations for nasal delivery include those with, for example and without limitation, dextran or cyclodextran.

### Dosing Schedule

It will be appreciated that in certain aspects, the formulations of the invention may be given as a single dose schedule, or preferably, in a multiple dose schedule. A multiple dose schedule is one in which a primary course of delivery may be with 1 to 10 separate doses, is optionally followed by other doses given at subsequent time intervals required to maintain or reinforce the treatment. The dosage regimen will also, at least in part, be determined by the needs of the individual and the judgement of the practitioner.

The invention thus provides a tablet comprising the pharmaceutical formulation of the invention; a capsule comprising the pharmaceutical formulation of the invention, an injectable suspension comprising the pharmaceutical formulation of the invention, and a formulation for pulmonary delivery comprising the pharmaceutical formulation of the invention. The AT₁R blocker and the CCR2 pathway inhibitor may be delivered in the same formulation or may be delivered in separate formulations.

The AT₁R blocker and the CCR2 pathway inhibitor may be in the same dosage form or may be in separate dosage forms. The subject being administered the AT₁R blocker and the CCR2 pathway inhibitor may be already receiving one of the active agents and may, in accordance with the present invention, be administered the other component of the treatment of the present invention. The treatment of the present invention may comprise the administration of only a CCR2 pathway inhibitor.

### Use

The invention also provides for the use of a pharmaceutical formulation comprising at least one chemokine receptor 2 (CCR2) pathway inhibitor for the manufacture of a formulation for the treatment, amelioration or prevention of ARDS.

The present invention further provides at least one CCR2 pathway inhibitor, for use in a formulation for the treatment, amelioration or prevention of ARDS.

The invention also provides for the use of a pharmaceutical formulation comprising (a) at least one angiotensin type 1 receptor (AT₁R) blocker, and (b) at least one chemokine receptor 2 (CCR2) pathway inhibitor for the manufacture of a formulation for the treatment, amelioration or prevention of ARDS.

The present invention further provides at least one AT₁R blocker, and at least one CCR2 pathway inhibitor, for use in a formulation for the treatment, amelioration or prevention of ARDS.

The present invention further provides at least one AT₁R blocker for use in a formulation for the treatment, amelioration or prevention of ARDS wherein the at least one AT₁R blocker is administered to the subject concurrently or sequentially with at least one CCR2 pathway inhibitor.

The present invention further provides at least one CCR2 pathway inhibitor for use in a formulation for the treatment, amelioration or prevention of ARDS wherein the at least one CCR2 pathway inhibitor is administered to the subject concurrently or sequentially with at least one AT₁R blocker.

Preferably the formulation is for use in the treatment, amelioration or prevention of ARDS. Preferably the ARDS is infection related ARDS. For example, the ARDS may be associated with infection by severe acute respiratory syndrome coronavirus 2 (SARS-CoV-2).

The CCR2 pathway inhibitor and the AT₁R blocker may be administered: in the same dosage form or in separate dosage forms. The CCR2 pathway inhibitor and the AT₁R blocker may be administered: concurrently or sequentially.

The CCR2 pathway inhibitor and/or the AT₁R blocker may be pharmaceutically acceptable salts of the CCR2 pathway inhibitor and/or the AT₁R blocker.

### Kits

The present invention provides a kit for the treatment or prevention of ARDS, said kit comprising:

a) at least one chemokine receptor 2 (CCR2) pathway inhibitor; and

b) instructions for use.

The present invention provides a kit for the treatment or prevention of ARDS, said kit comprising:

a) at least one angiotensin type 1 receptor (AT₁R) blocker;

b) at least one chemokine receptor 2 (CCR2) pathway inhibitor; and

c) instructions for use.

The contents of the kit can be lyophilized, and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the formulation may be applied to an affected area of the animal, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg.

Size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent, or may encompass two or more active agents.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

### Example 1: Clinical Study Examining the Effect of a CCR2 Antagonist in Patients with Community-Acquired Pneumonia (CAP)

The study may be a stand-alone study of a CCR2 antagonist or may be part of a larger study testing other therapeutics, including an angiotensin receptor blocker.

One example may be a multi-centre, adaptive, open-label or placebo-controlled study that recruits participants admitted to hospital with CAP. The treatment arm for a CCR2 antagonist expected to be approximately 500 patients.

Another example may be a multi-centre, multiple-arm, parallel assignment, randomized, open-label or placebo-controlled study that recruits patients admitted to hospital with CAP. This study would have approximately 500 to 1000 patients per treatment arm.

TABLE 2
Study of the effect of a CCR2 antagonist in patients with community-acquired pneumonia

| | |
|---|---|
| Study Title: | Global study examining the effect of a CCR2 antagonist in patients with |
| | community-acquired pneumonia. |
| Investigational | CCR2 antagonist, including propagermanium |
| Medicinal Product: | |
| Indication: | ARDS or CAP |
| Development Phase: | Phase 3 |
| Primary objective: | To evaluate the: |
| | All-cause mortality at 90 day in patients with CAP |
| Secondary | To evaluate the: |
| objective: | Effect of treatment with propagermanium on clinical outcomes. |
| | Effect of treatment on inflammatory markers |
| | Pharmacokinetics of CCR2 antagonist in patients with CAP |
| Primary endpoints: | To evaluate the: |
| | all-cause mortality at 90 days from enrolment |
| Secondary | To evaluate the: |
| endpoints: | In-hospital outcomes, and post-hospital outcomes including survival, HRQoL |
| | and Disability status at 6 months after enrolment |
| | Change in inflammatory biomarkers from enrolment |
| | Standard pharmacokinetic parameters |
| Study Design | The study is designed as a multicentre, open-label, randomised study evaluating |
| | the all-cause mortality at 90 days in patients with CAP. |
| | Approximately 500 patients with ARDS who meet all of the inclusion criteria and |
| | none of the exclusion criteria during screening and baseline assessments will be |
| | enrolled in this study from approximately 50 study sites globally. |
| | Patients may be receiving standard of care medication and may or may not be |
| | receiving concurrent dosing of an ARB (maximum tolerable dose) throughout the |
| | study. |
| | Measurements of patient health and treatment efficacy including biomarkers of |
| | inflammation will be performed during the study. |
| | Patients will initially be assessed for eligibility in a screening visit. |
| | Eligible patients will be randomised to either: |
| | Treatment Group 1: 120 mg propagermanium twice daily, OR |
| | Treatment Group 2: 120 mg propagermanium twice daily given concurrently |
| | with the maximum tolerated dose of an ARB, OR |
| | Treatment Group n: an alternative candidate selected by the Lead |
| | Investigator |
| | Patients will be monitored throughout the study for safety and efficacy |
| | assessments. |
| Number of patients: | The sample size for the assessment of CCR2 inhibition will be determined |
| | adaptively and frequently through the trial process based on the statistical |
| | assessment of treatment response. Nominally, this may include approximately |
| | 500 patients randomised to each treatment arm. |
| Number of centres | Approximately 50 centres |
| Inclusion criteria | 1. Adult patient admitted to with CAP within 48 hours of hospital admission with |
| | a. symptoms or signs or both that are consistent with lower respiratory tract |
| | infection (for example, acute onset of dyspnea, cough, pleuritic chest pain) AND |
| | b. Radiological evidence of new onset infiltrate of infective origin (in patients with |
| | pre-existing radiological changes, evidence of new infiltrate) |
| Exclusion criteria: | 1. Healthcare-associated pneumonia: a. Prior to this illness, is known to have |
| | been an inpatient in any healthcare facility within the last 30 days. |
| | 2. Death is deemed to be imminent and inevitable during the next 24 hours AND |
| | one or more of the patient, substitute decision maker or attending physician are |
| | not committed to full active treatment |
| | 3. Previous participation in this study within the last 90 days |
| | The Primary Endpoint for the study will be all-cause mortality at 90 days. The |
| | Secondary Endpoints will include in-hospital outcomes, and post-hospital |
| | outcomes including survival, HRQoL and Disability status at 6 months after |
| | enrolment and additional specific endpoints such as change in inflammatory |
| | biomarkers and pharmacokinetics. |
| Investigational | Propagermanium - 120 mg twice daily (total daily dose 240 mg) with or without |
| Medicinal Products: | concurrent dosing with the maximum tolerable dose of an ARB. Drug may be |
| | administered as oral capsules or in solution via gastric feeding tube. |
| Comparator: | Alternative therapies selected by Lead Investigator |
| Duration of | Up to 90 days |
| treatment: | |
| Results | A successful treatment for CAP patients with a CCR2 antagonist may include a |
| | statistical or clinical benefit in the primary or secondary outcomes of mortality, |
| | hospital outcomes, or inflammatory biomarker response, or a clinical benefit in |
| | other patient reported or health outcomes determined as medically important in |
| | the outcome for patients with CAP. |

### Example 2: Clinical Study Examining the Effect of a CCR2 Antagonist in Patients with Community-Acquired Pneumonia (CAP) Who are Released from Hospital

Patients who survive acute respiratory distress syndrome (ARDS) often leave a hospital intensive care unit with debilitating lung problems that may limit their quality of life. The study assesses the effect of a CCR2 antagonist in this population. The study may be a stand-alone study of a CCR2 antagonist or may be part of a larger study testing other therapeutics, including an angiotensin receptor blocker.

One example may be a multi-centre, open-label or placebo-controlled study that recruits participants released from hospital following CAP. The treatment arm for a CCR2 antagonist expected to be approximately 500 to 1000 patients.

TABLE 3
Study of the effect of a CCR2 antagonist in patients recovering from
ARDS associated with COVID-19 and following release from hospital

| | |
|---|---|
| Study Title: | Global study examining the effect of a CCR2 antagonist in patients recovering |
| | from community-acquired pneumonia and following release from hospital. |
| Investigational | CCR2 antagonist, including propagermanium |
| Medicinal Product: | |
| Indication: | ARDS or CAP |
| Development Phase: | Phase 3 |
| Primary objective: | To evaluate the: |
| | Lung function compared to baseline (on hospital release) and quality of life |
| | following CAP recovery |
| Secondary | To evaluate the: |
| objective: | Effect of treatment with propagermanium on clinical outcomes. |
| | Effect of treatment on inflammatory markers |
| | Pharmacokinetics of CCR2 antagonist in patients following CAP |
| Primary endpoints: | To evaluate the: |
| | Number of patients who demonstrate an increase in lung function |
| Secondary | To evaluate the: |
| endpoints: | Post-hospital outcomes, and post-hospital outcomes including HRQoL and |
| | Disability status at 6, 12 and 24 months after enrolment |
| | Change in inflammatory biomarkers from enrolment |
| | Standard pharmacokinetic parameters |
| Study Design | The study is designed as a multicentre, open-label, randomised study |
| | evaluating the lung function and quality of life following CAP recovery. |
| | Approximately 500 patients released from hospital following recovery from |
| | CAP who meet all of the inclusion criteria and none of the exclusion criteria |
| | during screening and baseline assessments will be enrolled in this study from |
| | approximately 50 study sites globally. |
| | Patients may be receiving standard of care medication and may or may not be |
| | receiving concurrent dosing of an ARB (maximum tolerable dose) throughout |
| | the study. |
| | At each visit, the patient's acute health status is reviewed to ensure |
| | appropriateness for pulmonary function testing, as well as completing a quality |
| | of life questionnaire. |
| | Measurements of patient health and treatment efficacy including biomarkers of |
| | inflammation and lung functions tests will be performed during the study. |
| | Patients will be monitored throughout the study for safety and efficacy |
| | assessments. |
| Results | A successful treatment for CAP patients may be a statistical or clinical benefit |
| | in lung function compared to baseline (upon release from hospital) and in |
| | quality of life. |

### Example 3: Clinical Study Examining the Effect of a CCR2 Antagonist in Patients with Community-Acquired Pneumonia (CAP) Prior to Hospitalisation

The study assesses the effect of a CCR2 antagonist on the recovery of patients upon diagnosis of CAP prior to hospitalisation, and the impact on the duration of CAP. The study may be a stand-alone study of a CCR2 antagonist or may be part of a larger study testing other therapeutics, including an angiotensin receptor blocker.

One example may be a multi-centre, open-label or placebo-controlled study that recruits participants diagnosed with CAP. The treatment arm for a CCR2 antagonist expected to be approximately 500 to 1000 patients.

TABLE 4
Study of the effect of a CCR2 antagonist in patients diagnosed
with ARDS associated with COVID-19 prior to hospitalisation

| | |
|---|---|
| Study Title: | Global study examining the effect of a CCR2 antagonist in patients diagnosed |
| | with community-acquired pneumonia prior to hospitalisation. |
| Investigational | CCR2 antagonist, including propagermanium |
| Medicinal Product: | |
| Indication: | ARDS or CAP |
| Development Phase: | Phase 3 |
| Primary objective: | To evaluate the: |
| | Duration of CAP, including recovery time |
| | Lung function |
| | Quality of life following CAP recovery |
| Secondary | To evaluate the: |
| objective: | Effect of treatment with propagermanium on clinical outcomes. |
| | Effect of treatment on inflammatory markers |
| | Pharmacokinetics of CCR2 antagonist in patients following CAP |
| Primary endpoints: | To evaluate the: |
| | The number of patients who do not require hospitalisation versus those that |
| | do; and |
| | The number of patients who see an increase in lung function compared to |
| | baseline (diagnosis); |
| | A reduction in recovery time versus placebo |
| Secondary | To evaluate the: |
| endpoints: | Post-hospital outcomes, and post-hospital outcomes including survival, |
| | HRQoL and Disability status at 6, 12 and 24 months after enrolment |
| | Change in inflammatory biomarkers from enrolment |
| | Standard pharmacokinetic parameters |
| Study Design | The study is designed as a multicentre, open-label, randomised study evaluating |
| | the lung function and quality of life following CAP recovery. |
| | Approximately 500 patients released from hospital following recovery from CAP |
| | who meet all of the inclusion criteria and none of the exclusion criteria during |
| | screening and baseline assessments will be enrolled in this study from |
| | approximately 50 study sites globally. |
| | Patients may be receiving standard of care medication and may or may not be |
| | receiving concurrent dosing of an ARB (maximum tolerable dose) throughout the |
| | study. |
| | At each visit, the patient's acute health status is reviewed to ensure |
| | appropriateness for pulmonary function testing, as well as measurements of |
| | patient health and treatment efficacy including biomarkers of inflammation and |
| | lung functions tests will be performed during the study. |
| | Patients will be monitored throughout the study for safety and efficacy |
| | assessments. |
| Results | A successful treatment for CAP patients may be a reduced number of patients |
| | requiring hospitalisation; and a statistical or clinical benefit in lung function |
| | compared to baseline (upon diagnosis). |

### Example 4: Clinical Study Examining the Effect of a CCR2 Antagonist in Patients with COVID-19 Respiratory Complications, Prior to the Onset of Acute Respiratory Distress Syndrome (ARDS)

The study is a Phase 2/3 assessment of the effect of a CCR2 antagonist on patients with COVID-19 respiratory complications, prior to the onset of Acute Respiratory Distress Syndrome (ARDS). The study may be a stand-alone study of a CCR2 antagonist or may be part of a larger study testing other therapeutics, including an angiotensin receptor blocker.

One example may be a prospective, multi-centre, randomised, double blind, placebo-controlled study that recruits participants diagnosed with COVID-19 respiratory complications. The treatment arm for a CCR2 antagonist is expected to be approximately 600 patients.

The aim of this study is to evaluate the safety and efficacy of dual treatment with repagermanium and an AT₁R blocker compared to placebo for patients hospitalised for management of COVID-19.

The safety and efficacy of dual treatment with repagermanium and candesartan will be assessed by the Clinical Health Score at day 14. This Clinical Health Score is determined within a 7-point ordinal scale of health status which is a modified version of the 9-point score developed by the WHO for Coronavirus Disease 2019 (COVID-19) trials. A single score will be reported with higher values corresponding to worse symptoms. The ordinal scale is an assessment of the clinical status of the participant at the first assessment for the day, measured at Day 14 after the date of randomisation.
7-point ordinal scale of health status:
1. Not hospitalised, no limitations on activities.
2. Not hospitalised, limitation on activities.
3. Hospitalised, not requiring supplemental oxygen.
4. Hospitalised, requiring supplemental oxygen.
5. Hospitalised, on non-invasive ventilation or high-flow oxygen devices.
6. Hospitalised, on invasive mechanical ventilation or ECMO.
7. Death.

The secondary objectives are to evaluate the safety and efficacy of dual treatment with repagermanium and candesartan in patients hospitalised with COVID-19 disease, assessed by:
Clinical Health Score at day 28.
ICU admission (incidence in days 0-28).
Death (incidence in days 0-28).
Time to death, assessed from hospital admission to death.
Acute Kidney Injury (incidence in days 0-28).
Respiratory Failure (incidence in days 0-28).
Length of hospital admission (days of inpatient stay from admission to discharge or death).
Length of ICU Admission (days in ICU from admission to transfer to ward or death).
Requirement of ventilatory support (count of days with ventilation in days 0-28).
Requirement of dialysis (count of days with dialysis in days 0-28).
Clinical Health Score at day 60.
Clinical Health Score at day 90.
Clinical Health Score at day 180.

The specific safety objectives are to evaluate the safety of dual treatment with epagermanium and candesartan in patients hospitalised with COVID-19 disease, assessed by incidence of pre-specified clinical events:
Hypotension, requiring an urgent or non-urgent intervention (e.g., reduction in dose or cessation of anti-hypertensive, vasopressors, intravenous fluids). Incidence in days 0-28.
Hyperkalaemia (defined as a K>5.5-6.0 mmol/L or requiring an intervention including hospitalisation; K>6.0 mmol/L). Incidence in days 0-28.

The exploratory objective is to evaluate the effect of dual treatment with repagermanium and candesartan in patients hospitalised with COVID-19 disease on hospital readmission rate, assessed by incidence of hospital readmission. Admission for overnight stay up to day 90 following initial hospital discharge.

### Design

CLARITY 2.0 is a prospective, Multi-Centre, Multi-Arm Multi-Stage Randomised, Double Blind, Placebo Controlled Phase II Trial, utilising adaptive sample size re-estimation. Stage 1 will include approximately 600 participants prior to a review of accumulated data and expansion to Stage 2 Phase III study.

Participants will be randomised into three treatment arms:

1. Candesartan+repagermanium, (Investigational Arm);

2. Candesartan+placebo [repagermanium], (Control Arm #1); and

3. Placebo [candesartan]+placebo [repagermanium] (Control Arm #2) initially in a 1:1:1 ratio.

### Study Population

Participants must meet all the inclusion criteria, and none of the exclusion criteria, to be eligible for this trial.

### Target Population

Adults with laboratory-confirmed SARS-CoV-2 infection intended for hospital admission for management of COVID-19.

### Eligibility Inclusion Criteria

a. Adults aged 18 years
b. Laboratory-confirmed diagnosis of SARS-CoV-2 infection within 10 days prior to randomisation. (Confirmation through appropriate approved laboratory or Point of Care testing method, including Polymerase Chain Reaction (PCR) or other public health assay.)

c. Intended for hospital admission for management of COVID-19.

d. Systolic Blood Pressure (SBP)≥120 mmHg OR SBP≥115 mmHg and currently treated with a non-RAASi BP lowering agent that can be ceased.

### Eligibility Exclusion Criteria

1. Currently treated with an ACEi, ARB or aldosterone antagonist, aliskiren, or ARNi
2. Intolerance of ARBs
3. Serum potassium >5.5 mmol/L
4. An estimated Glomerular Filtration Rate (eGFR)<30 ml/min/1.732 m
5. Known biliary obstruction, known severe hepatic impairment (Child-Pugh-Turcotte score 10-15)

### Randomisation

Participants will be randomised by their treating team. Through the randomisation process the participant will be assigned a treatment arm. Treatment should be commenced as soon as possible and within one day after randomisation. Participants will be encouraged to continue in the trial, even if they withdraw from the randomised treatment allocation, to enable intention-to-treat analyses. Initially, treatment allocation will be with a 1:1:1 block randomisation between three arms, stratified by centre.

### Treatment Plan

### Treatments

The intervention being investigated in this trial is a dual therapy of candesartan and repagermanium.

Candesartan is an ARB. ARB treatment is commonly used in the management of hypertension, or for the prevention of progression of diabetic kidney disease or secondary prevention of cardiovascular events.

Repagermanium is a CCR2 antagonist with an extensive safety database and, unlike many immune modulators, has a low AE profile. When administered concurrently with an ARB, repagermanium is designed to inhibit recruitment of monocytes via both the AT₁R and CCR2 pathways implicated in the inflammatory cytokine environment of respiratory distress.

### Study Treatment

Participants will be randomized to one of three arms described in Table 1, including repagermanium, or matched placebo, and candesartan, or matched placebo.

TABLE 4
Trial Arms treatment

| | | | |
|---|---|---|---|
| Agent | Commencing dose | Route | Duration |

Investigational Arm Interventions

| | | | |
|---|---|---|---|
| Candesartan | Titratable commencing dose 4 mg | Oral | 28 days |
| | tablets twice daily (daily dose | | |
| | 8 mg) | | |
| Repagermanium | 120 mg immediate release capsule | Oral | |
| | twice daily (daily dose 240 mg) | | |

Control Arm #1 Interventions

| | | | |
|---|---|---|---|
| Candesartan | Titratable commencing dose 4 mg | Oral | 28 days |
| | tablets twice daily (daily dose | | |
| | 8 mg) | | |
| Placebo | Matching capsules twice daily | Oral | |
| [Repagermanium] | | | |

Control Arm #2 Interventions

| | | | |
|---|---|---|---|
| Placebo | Titratable tablets twice daily | Oral | 28 days |
| [Candesartan] | | | |
| Placebo | Matching capsules twice daily | Oral | |
| [Repagermanium] | | | |

The dose of repagermanium will be delivered as 120 mg capsules given twice a day. The dose will not be changed during the trial. The starting dose of candesartan will be 4 mg given twice a day. The dose will be titrated in accordance with the algorithm presented in FIG. 2.

### Respiratory & COVID-19 Care

For patients within this trial, background care and respiratory management will be according to local site regulations, recommendations and protocol in the setting of the pandemic. For patients within this trial, background care must include local investigations of Blood Pressure, Potassium and Renal Function as directed within the schedule of assessments.

Patient safety is the primary responsibility of the treating clinician, as such their discretion should determine the course of action for the management of toxicities. In general, treatment should be withheld during AEs of severity Grade 3-4 (according to the Common Terminology Criteria for Adverse Events (CTCAE)), and not restarted until the AE has resolved to Grade 0-1, at the investigator's discretion.

### Concomitant Medications

The following medications should not be used during this study.
i. Other novel investigational treatments—patient is ineligible.
ii. Any ACEis—patient is ineligible.
iii. Any other ARBs—patient is ineligible.
iv. Aldosterone antagonist—patient is ineligible.
v. Aliskiren—patient is ineligible.
vi. ARNi—patient is ineligible.
vii. NSAIDs—patients should be advised not to take NSAIDs whilst they are receiving treatment in the trial. This includes Nurofen, Ibuprofen, Advil, Voltaren and Mobic. Use of Paracetamol or Aspirin is permitted.

Any other BP-lowering medication can continue except in the setting of hypotension.

### Compliance

Participants and trial sites are encouraged to maintain appropriate treatment with the study intervention without interruption. Where treatment is missed or discontinued for any reason, relevant information should be documented. An intention-to-treat analysis will be performed on this trial data.

### Screening

Potential participants will be screened within the 10 days prior to randomisation to ensure they meet all the inclusion criteria and do not meet any of the exclusion criteria as described above. Assessments necessary to ascertain eligibility include SARS-CoV-2 test within 10 days prior to randomisation, blood pressure, serum potassium (test performed or on record within the 3 months prior to randomisation) and eGFR (test performed or on record within the 3 months prior to randomisation).

### Baseline Assessment

Demographics and medical history assessment including details of the SARS-CoV-2 test and results, blood pressure, electrocardiogram (ECG), date of birth, sex, weight, height, smoking history, pregnancy test status (for women younger than 51 years), comorbidities, concomitant medications, and most recent blood test results. Ethnicity will be recorded.

### Primary Follow Up & Treatment Period (Day 1 to 28)

Participants will be provided with their randomised interventions following randomisation. Treatment will continue for 28 days. Additional Candesartan may be supplied at any time in the first 28 days via resupply to accommodate for up titration in dose.

Participants will be followed up daily for 28 days after randomisation. This follow-up includes:

i. Health status assessment including assessment of hospital admission status, ventilation status, supplemental oxygen status, intensive care unit status, and mortality status and collection of concomitant medications will be undertaken from baseline to day 28 from commencement of study treatment.
ii. Blood pressure will be assessed from baseline to day 14 from commencement of study treatment.
iii. Blood tests will be undertaken at baseline, then days 1, 3, 5, 7, 9 and 14 from commencement of study treatment. Blood tests will include Potassium, Serum Creatinine, Creatinine Kinase, estimated Glomerular Filtration Rate (using CKD-EPI creatinine equation), White Blood Cell Count, Neutrophil Count, Lymphocyte Count, D-Dimer, C-Reactive Protein, Aspartate Aminotransferase and Alanine Aminotransferase.
iv. Safety events will be reportable from initiation of trial treatment until day 60.

### Secondary Follow-Up (Day 29 to 180)

Participants will be followed up for 180 days after randomisation to reach the study endpoint. The health status assessment measurement will be undertaken at 60 days, 90 days and 182 days and can be done by phone or clinic visit. Each visit will occur within a 7-day window of the scheduled visit date. Safety events will be reportable from initiation of trial treatment until day 60.

### Outcomes, Endpoints and Other Measurements

### Primary Endpoint Definition

The primary endpoint is a Clinical Health Score, determined within a 7-point ordinal scale of clinical health status as described above which is a modified version of the 9-point score developed by the WHO for Coronavirus Disease 2019 (COVID-19) trials. A single score will be reported with higher values corresponding to worse symptoms. The ordinal scale is an assessment of the clinical status of the participant at the first assessment for the day, measured at Day 14 after the date of randomisation.

Clinical assessment includes review of hospital medical records, following admission by the investigator, for Clinical Health Score events at day 14 (including hospitalisation, use of oxygen, ventilation, ECMO or death due to any cause). Where the participant has been discharged, Clinical Health Score events will be confirmed by the participant (by phone or clinic visit) or local General Practitioner. Community-based death events will be confirmed with the participant's local General Practitioner in local medical records.

### Secondary Endpoint Definitions

### Clinical Health Score at Day 28

Clinical assessment includes review of hospital medical records, following admission by the investigator, for Clinical Health Score events at day 28 (including hospitalisation, use of oxygen, ventilation, ECMO or death due to any cause). Where the participant has been discharged, Clinical Health Score events will be confirmed by the participant (by phone or clinic visit) or local General Practitioner. Community-based death events will be confirmed with the participant's local General Practitioner in local medical records.

### Intensive Care Unit (ICU) Admission

Incidence in days 0-28. Clinical assessment includes review of hospital medical records following admission by the investigator or notification of admission by the participant or local General Practitioner for ICU admission (for any reason).

### Death

Incidence in days 0-28. Clinical assessment includes review of hospital medical records following admission by the investigator or notification of death (due to any cause) by the participant's local General Practitioner. Community-based death events will be confirmed with the participant's local General Practitioner in local medical records.

### Time to Death

Assessed from hospital admission to death. Clinical assessment includes review of hospital medical records following admission by the investigator or notification of death (due to any cause) by the participant's local General Practitioner. Community-based death events will be confirmed with the participant's local General Practitioner in local medical records.

Acute Kidney Injury (AKI)

Incidence in days 0-28. Clinical assessment includes review of hospital medical records following admission by the investigator or notification of AKI by the participant or local General Practitioner. AKI is defined by the KDIGO criteria as an increase in serum creatinine by ≥0.3 mg/dL within 48 hours or an increase in serum creatinine ≥1.5 times from baseline within the last 7 days or urine output <0.5 mL/kg/h for 6 hours. Community-based AKI events will be confirmed with the participant's local General Practitioner in local medical records.

### Respiratory Failure

Incidence in days 0-28. Clinical assessment includes review of hospital medical records following admission by the investigator or notification of respiratory failure events by the participant or local General Practitioner. Community-based respiratory failure events will be confirmed with the participant's local General Practitioner in local medical records.

### Length of Hospital Admission

Days of inpatient stay from admission to discharge or death. Clinical assessment includes review of hospital medical records following admission by the investigator or notification of hospital admission by the participant or local General Practitioner.

### Length of ICU Admission

Days in ICU from admission to transfer to ward or death. Clinical assessment includes review of hospital medical records following admission by the investigator or notification of overnight admissions to ICU by the participant or local General Practitioner.

### Requirement of Ventilatory Support

Count of days with ventilation in days 0-28. Clinical assessment includes review of hospital medical records following admission by the investigator or notification of episodes of ventilatory support by the participant or local General Practitioner. Ventilatory support is defined as the delivery of oxygen via invasive or non-invasive ventilation.

### Requirement of Dialysis

Count of days with dialysis in days 0-28. Clinical assessment includes review of hospital medical records following admission by the investigator or notification of episodes of dialysis support by the participant or local General Practitioner. Dialysis support is defined as any occurrence of haemodialysis or peritoneal dialysis. Community-based dialysis will be confirmed with the participant's local General Practitioner in local medical records.

### Clinical Health Score at Day 60

Clinical assessment includes phone or clinic visit for Clinical Health Score events at day 60, including hospital admission (for any reason) or death (due to any cause). Community-based death events will be confirmed with the participant's local General Practitioner in local medical records.

### Clinical Health Score at Day 90

Clinical assessment includes phone or clinic visit for Clinical Health Score events at day 90, including hospital admission (for any reason) or death (due to any cause). Community-based death events will be confirmed with the participant's local General Practitioner in local medical records.

### Clinical Health Score at Day 180

Clinical assessment includes phone or clinic visit for Clinical Health Score events at day 180, including hospital admission (for any reason) or death (due to any cause). Community-based death events will be confirmed with the participant's local General Practitioner in local medical records.

### Safety Objectives and Measures

### Hypotension

Incidence in days 0-28. Clinical assessment includes review of hospital medical records following admission by the investigator or notification of hypotensive events (requiring an urgent or non-urgent intervention) by the participant or local General Practitioner. Hypotension is defined as blood pressure readings <90 mmHg systolic or <60 mmHg diastolic; urgent or non-urgent intervention includes, but is not limited to, reduction in dose or cessation of anti-hypertensives, vasopressors, intravenous fluids. Community-based hypotensive events will be confirmed with the participant's local General Practitioner in local medical records.

### Hyperkalaemia

Incidence in days 0-28. Clinical assessment includes review of hospital medical records following admission by the investigator or notification of hyperkalemic events by the participant or local General Practitioner. Hyperkalaemia is defined as a K>5.5-6.0 mmol/L and requiring an intervention including hospitalisation, or K>6.0 mmol/L. Community-based hyperkalemic events will be confirmed with the participant's local General Practitioner in local medical records/pathology reports.

### Deranged Liver Function Tests (LFTs)

Incidence in days 0-28. Incidence in days 0-28. Clinical assessment includes review of hospital medical records following admission by the investigator or notification of deranged LFTs by the participant or local General Practitioner. Deranged LFTs is defined as ALT or AST >ULN or >1.5 times baseline. Community based LFT derangement will be confirmed with the participant's local General Practitioner in local medical records/pathology reports.

### Exploratory Objective and Measures

### Incidence of Hospital Readmission

Admission for overnight stay up to day 90 following initial hospital discharge. Clinical assessment includes review of hospital medical records to day 90 from primary admission at randomisation.

### Study Treatment Supply

### Investigational Arm Treatment

Participants will receive candesartan 4 mg tablets and repagermanium 120 mg capsules in the study.

Candesartan will be commenced at a daily dose of 8 mg, titrated in accordance with the algorithm presented in FIG. 2. Repagermanium will be commenced at a fixed daily dose of 240 mg.

### Control Arms Treatment

Participants will receive:
a. Candesartan 4 mg tablets and placebo repagermanium capsules. Candesartan will be commenced at a daily dose of 8 mg, titrated in accordance with the algorithm presented in FIG. 2. Placebo repagermanium capsules will be commenced at the fixed twice daily regimen.

### OR

b. Placebo [candesartan] tablets and Placebo repagermanium capsules. Candesartan placebo tablets will be commenced at a twice daily regimen, titrated in accordance with the algorithm presented in FIG. 2. Placebo [repagermanium] capsules will be commenced at the fixed twice daily regimen.

### Treatment Administration

The investigational medicinal product (IMP) will be self-administered, within an hour of eating food, by participants who will be directed to take the IMP each day at approximately the same time (e.g., 9:00 am and 7:00 pm±1 hour).

### Statistical Considerations

### Sample Size

Stage 1 of the trial will recruit 600 participants.

A review of the accumulated data following Stage 1 of the trial, together with data accumulated in other trials, will inform the decision to transition into Stage 2 of the Phase III trial. Stage 2 will use the accumulated evidence to support selection of the primary comparator.

Stage 2 of the trial will be conducted using Bayesian methods, with rules determined from simulated analyses for stopping rules on efficacy, futility, and harm. Adaptive sample size estimation will be employed and detailed within an updated statistical considerations section, following completion of trial simulations.

### Statistical Analysis

This study will use Bayesian principles and Monte Carlo methods to evaluate the outcomes of interest. All inferences will be based on posterior distributions of the model parameters. Trial decisions will be based on the posterior and predictive distributions.

The estimated treatment effect on the primary endpoint will be expressed as the common odds ratio, corresponding to the odds of a better outcome in the investigational arm versus each comparator arm on the 7-point ordinal scale at Day 14, and its 95% credible interval. This will be modelled using a proportional odds logistic regression model. Details will be provided in the statistical analysis plan which will be completed prior to any analysis of the data.

The assessment of the primary endpoint at Day 14 reflects the timeframe within which most SARS-CoV-2-positive patients have either developed severe disease or begun to recover.

The primary analysis will be an ITT analysis, whereby comparisons will be made between all participants randomized to the treatment arms and who have passed the primary endpoint, irrespective of whether they received their allocated treatment.

To explore the power achieved with the sample size, extensive simulations will be performed under various assumptions. Details of these simulations and their underlying assumptions will be provided in the statistical analysis plan.

### REFERENCES

Ayoub M A, et al., 2015. Functional interaction between angiotensin II receptor type 1 and chemokine (C-C motif) receptor 2 with implications for chronic kidney disease. PLoS ONE 10: e0119803. https://doi.org/10.1371/journal.pone.0119803
Huang C, et al. 2020. Clinical features of patients infected with 2019 novel coronavirus in Wuhan, China. Lancet 395: 497-506. https://doi.org/10.1016/S0140-6736(20)30183-5
Cheng Y, et al. 2020. Kidney disease is associated with in-hospital death of patients with COVID-19. [published online ahead of print Mar. 20, 2020]. Kidney International. https://doi.org/10.1016/j.kint.2020.03.005
Fang L, et al., 2020. Lancet Respiratory Medicine. V8, e21. https://www.thelancet.com/pdfs/journals/lanres/PIIS2213-2600(20)30116-8.pdf
Kuba K, et al., 2005 A crucial role of angiotensin converting enzyme 2 (ACE2) in SARS coronavirus-induced lung injury. Nat Med 11(8): 875-879, https://doi.org/10.1038/nm1267,
Yi et al., 2019. Monocyte Chemoattractant Protein-1, a Possible Biomarker of Multiorgan Failure and Mortality in Ventilator-Associated Pneumonia. Int J Mol Sci 20: 2218. https://doi.org/10.3390/ijms20092218
Sanchis-Gomar F, et al., 2020. Angiotensin-converting enzyme 2 and anti-hypertensives (angiotensin receptor blockers and angiotensin converting enzyme inhibitors) in coronavirus disease 2019 (COVID-19) [published online ahead of print Mar. 30, 2020]. Mayo Clin Proc. http://doi.org/10.1016/j.mayocp.2020.03.026
Sriram K & Insel P, 2020. Dangers of ACE inhibitor and ARB usage in COVID-19: evaluating the evidence. medRxiv 2020.03.25.20043927; doi:https://doi.org/10.1101/2020.03.25.20043927
Wong C K et al., 2004. Plasma inflammatory cytokines and chemokines in severe acute respiratory syndrome. Clin Exp Immunol 136: 95-103. https://doi.org/10.1111/j.1365-2249.2004.02415.x
Xiong Y et al., 2020. Transcriptomic characteristics of bronchoalveolar lavage fluid and peripheral blood mononuclear cells in COVID-19 patients. Emerging Microbes & Infections, 9:1, 761-770 https://doi.org/10.1080/22221751.2020.1747363
Yang G, et al., 2020. Angiotensin II Receptor Blockers and Angiotensin-Converting Enzyme Inhibitors Usage is Associated with Improved Inflammatory Status and Clinical Outcomes in COVID-19 Patients With Hypertension. medRxiv 2020.03.31.20038935; doi:https://doi.org/10.1101/2020.03.31.20038935
Sanchis-Gomar, F et al 2020. Angiotensin-Converting Enzyme 2 and Antihypertensives (Angiotensin Receptor Blockers and Angiotensin-Converting Enzyme Inhibitors) in Coronavirus Disease 2019 Mayo Clinic Proceedings https://dx.doi.org/10.1016/j.mayocp.2020.03.026
Yokochi, S. 2001. Journal of Interferon and Cytokine Research 21:389-398
van Zoelen M., et al 2011. Endogenous MCP-1 promotes lung inflammation induced by LPS and LTA. Mol Immunol. 2011 July; 48(12-13):1468-76. http://doi.org/10.1016/j.molimm.2011.04.001
Wang et al., 2019. Renin-angiotensin-system, a potential pharmacological candidate, in acute respiratory distress syndrome during mechanical ventilation. Pulm Pharmacol Ther. 2019 October; 58: 101833. Published online 2019 Aug. 1. doi: 10.1016/j.publ.2019.101833

1. A method for the treatment or amelioration of Acute Respiratory Distress Syndrome (ARDS) comprising the steps of:
administering to a subject a therapeutically effective amount of at least one chemokine receptor 2 (CCR2) pathway inhibitor or a pharmaceutically acceptable salt thereof.

2. A method for the treatment or amelioration of Acute Respiratory Distress Syndrome (ARDS) comprising the steps of:
administering to a subject a therapeutically effective amount of: a) at least one angiotensin 1 receptor blocker (AT₁R) or a pharmaceutically acceptable salt thereof, and b) at least one chemokine receptor 2 (CCR2) pathway inhibitor or a pharmaceutically acceptable salt thereof.

3. The method of claim 1 or 2 wherein the ARDS is infection related ARDS.

4. The method of claim 1 or 2 wherein the ARDS is associated with infection by severe acute respiratory syndrome coronavirus 2 (SARS-CoV-2).

5. The method of claim 2 wherein the AT₁R blocker is selected from the group consisting of: irbesartan; eprosartan; losartan; valsartan; telmisartan; candesartan; olmesartan; and ZD-7115.

6. The method of claim 1 or 2 wherein the CCR2 pathway inhibitor:
i) inhibits MCP-1 induced migration and activation of monocytes and chemotactic migration through the targeting of one or more glycosylphosphatidylinositol (GPI)-anchored proteins selected from the group consisting of CD55, CD59 and CD16;
and/or ii) stabilizes the complexes CCR2/CD55 and/or CCR2/CD59 and/or CCR2/CD16; and/or
iii) is propagermanium.

7. The method of claim 2 wherein the CCR2 pathway inhibitor and the AT₁R blocker are administered:
i) in the same dosage form;
ii) in separate dosage forms.

8. The method of claim 2 wherein the CCR2 pathway inhibitor and the AT₁R blocker are administered:
i) concurrently;
ii) sequentially.

9. The method of claim 1 or 2 wherein about 5 mg to 1 g of the CCR2 pathway inhibitor is administered in a dosage form.

10. The method of claim 2 wherein about 1 mg to 1200 mg of the AT₁R blocker is administered in a dosage form.

11. Use of at least one chemokine receptor 2 (CCR2) pathway inhibitor for the manufacture of a dosage form for the treatment or prevention of ARDS.

12. Use of (a) at least one angiotensin type 1 receptor (AT₁R) blocker, and (b) at least one chemokine receptor 2 (CCR2) pathway inhibitor for the manufacture of a dosage form for the treatment or prevention of ARDS.

13. A kit for the treatment, amelioration or prevention of ARDS, said kit comprising:
i) at least one chemokine receptor 2 (CCR2) pathway inhibitor; and
ii) instructions for use.

14. A kit for the treatment, amelioration or prevention of ARDS, said kit comprising:
i) at least one angiotensin type 1 receptor (AT₁R) blocker;
ii) at least one chemokine receptor 2 (CCR2) pathway inhibitor; and
iii) instructions for use.

15. At least one CCR2 pathway inhibitor, for use in a formulation for the treatment, amelioration or prevention of ARDS.

16. At least one AT₁R blocker, and at least one CCR2 pathway inhibitor, for use in a formulation for the treatment, amelioration or prevention of ARDS.

17. At least one AT₁R blocker for use in a formulation for the treatment, amelioration or prevention of ARDS wherein the at least one AT₁R blocker is administered to the subject concurrently or sequentially with at least one CCR2 pathway inhibitor.

18. At least one CCR2 pathway inhibitor for use in a formulation for the treatment, amelioration or prevention of ARDS wherein the at least one CCR2 pathway inhibitor is administered to the subject concurrently or sequentially with at least one AT₁R blocker.
